(19) United States
(12) Patent Application Publication
(10) Pub. No.: US 2003/0157145 A1
(43) Pub. Date: Aug. 21, 2003

(54) Fluoridated products

(76) Inventors: Tom K. Kalili, Beverly Hills, CA (US); Angelo A. Caputo, Calabasas, CA (US); Shawn Caley, Glendale, CA (US)

Correspondence Address: E. JOSEPH GESS, BURNS, DOANE, SWECKER & MATHIS, L.L.P., P.O. Box 1404, Alexandria, VA, 22313-1404, US

(21) Appl. No.: 10/346,068

(22) Filed: Jan. 17, 2003

Related U.S. Application Data
(63) Continuation-in-part of application No. 10/053,714, filed on Jan. 24, 2002.

(51) Int. Cl. A61K 31/59; A61K 31/34; A61K 31/375; A61K 47/00

(52) U.S. Cl. 424/439; 514/167; 514/474

(57) ABSTRACT

Provided are fluoridated products, including a beverage and a powdered composition for preparing the beverage as well as oral healthcare products. The powdered composition can be readily mixed with water to prepare a suitable fluoridated beverage or solution for making other products upon the addition of hydrogen fluoride. The powdered composition comprises a fluoride compound, an essential amino acid and a polyprotic acid, and most preferably vitamin C. A preferred method of preparing the fluoridated solution includes adding hydrogen fluoride to the water base, as the presence of the hydrogen fluoride greatly enhances the fluoride availability, and then adding the remaining components. The solution can be used as a fluoridated beverage or used to prepare other oral healthcare products which provide enhanced protection of tooth surfaces from cavities and eventual tooth loss.

### CROSS-REFERENCE TO RELATED APPLICATIONS

The present application is a continuation-in-part of U.S. Ser. No. 10/053,714 filed Jan. 24, 2002, which is incorporated herein in its entirety by reference.

### BACKGROUND OF THE INVENTION

1. Field of the Invention

The present invention relates to fluoridated products, including powdered compositions useful in making fluoridated beverages, the novel fluoridated beverages themselves, and oral healthcare products such as toothpaste. The present invention also relates to a method of making the fluoridated products.

2. Description of the Related Art

The effectiveness of fluoride in lowering the incidence of dental caries is of major significance to the entire field of dental health. The precise mechanism whereby fluoride reduces tooth decay remains unknown. Innovative avenues of investigation have theorized that structural chemical, physicochemical, electrochemical or thermodynamic mechanisms may be involved. However, at the present time, no single theory adequately explains all the phenomena of dental decay or the precise role which fluoride plays in reducing tooth destruction. It is believed that one possible mechanism for decay reduction is based on the characteristic of fluoride to cause remineralization of early enamel lesions, thereby rendering the tooth more resistant to subsequent decay.

Traditionally, adults do not benefit from fluoride since the porosity (prism layers) within the enamel surface of tooth structure constrict in size or calcify as we transition from adolescence to adulthood. As a result, researchers have investigated various fluoride delivery systems to improve fluoride efficacy.

Approaches have included incorporation of fluoride in drinking water, dental topicals and rinses, and toothpastes. Throughout all the investigations, it has been noted consistently that a direct relationship exists between availability and the uptake of appropriate levels of fluoride and a decrease of an incidence of dental decay. Based on this observation, fluoride administration has evolved as the foremost approach toward providing widespread protection against dental caries.

Various investigators have conducted studies to provide sufficient amount of fluoride in liquid compositions to control tooth decay and minimize the effect of fluorosis. Grodberg, U.S. Pat. No. 5,153,005 provides fluoride compounds in liquids, beverage additives, flavored drops of fluoride, etc. Luoma U.S. Pat. No. 2,230,116; Grigoriev, U.S. Pat. No. 2,035,880 and Blaser, U.S. Pat. No. 5,182,114, teach of liquid compositions such as beverage, bottled mineral water, fruit juice, etc. to provide concentrates having high contents of fluoride. U.S. Pat. Nos. 4,152,419; 4,425,324; 4,528,181 and British Patent No. 1,435,624 teach fluorosis-free compositions containing binary fluoride sources of sodium fluoride and sodium monofluorophosphate, and the American Dental Association in the accepted Dental Therapeutics, 38^{th} edition, lists liquid acidulated compositions containing an acid, and binary fluoride compounds in high concentrations intended only for rinsing of the oral cavity to control tooth decay. These compositions contain about 2.5 percent sodium fluoride, 0.35 percent hydrogen fluoride and 1.1 percent orthophosphoric acid. Ingestion of these concentrations will cause sublethal symptoms such as nausea, vomiting, abdominal distress, diarrhea, stupor, weakness and lethal symptoms such as muscular weakness, tremors, convulsions, collapse, dyspnea, respiratory, cardiac failure, and death.

A problem with utilizing a fluoride compound in an aqueous solution is the lasting availability of the fluoride. It is well known that when a soluble fluoride compound such as sodium fluoride is added to water, products are formed until the sodium fluoride has been completely used or an equilibrium has been reached. The fluoride ions, in reaction with water, form hydrogen fluoride (or hydrofluoric acid) and hydroxy ions. This greatly limits the availability of the fluoride ions in such a beverage/solution, and can lead to little if any fluoride available when the beverage is actually used.

Thus, it is an object of the present invention to provide a stable fluoride composition useful in preparing beverages, e.g., water based beverages.

Yet another object of the present invention is to provide a stable water based beverage which maximizes availability of fluoride ions.

Still another object of the present invention is to provide a safe and effective fluoridated beverage which provides optimal caries inhibition.

Another object of the present invention is to provide oral healthcare products prepared by using a novel, stable fluoridated solution.

These and other objects of the present invention will become apparent to the skilled artisan upon a review of the following description and the claims appended hereto.

### SUMMARY OF THE INVENTION

The foregoing objectives are realized by providing a composition comprised of a fluoride compound, an essential amino acid and a weak polyprotic acid. This composition can be in powder form so that it may be easily transported, and mixed with water in order to help prepare a useful fluoridated solution upon the addition of hydrogen fluoride to the aqueous solution. The composition preferably also contains ascorbic acid (vitamin C), as well as at least one of vitamin D, calcium and a phosphate compound.

In another embodiment, the present invention provides one with a fluoridated beverage comprising water, a fluoride compound, hydrogen fluoride and a polyprotic acid. The fluoridated beverage will have a reduced pH, i.e., in the range of from 2.0 to 7.0, and most preferably in the range of from 3.5 to 6.0. The reduced pH of the beverage allows for better penetration of the fluoride into teeth, while the use of a dual fluoride source, i.e., a fluoride compound such as stannous fluoride or sodium fluoride, and hydrogen fluoride, provides for a stable beverage with regard to maximized fluoride activity.

In a preferred embodiment, the beverage further comprises an essential amino acid such as lysine. Other components, such as vitamin C, vitamin D, calcium and/or a phosphate compound, can also be added. It is also preferred that the beverage is non-carbonated.

The fluoridated beverage in accordance with the present invention is prepared by adding to water the hydrogen fluoride, the fluoride compound, polyprotic acid, and any other desired components, such as lysine and vitamin C. In a most preferred embodiment, citric fruit juices are used to provide a weak polyprotic acid, i.e., citric acid, as well as vitamin C (ascorbic acid). The citric fruit juices would also provide flavoring to the beverage.

In another embodiment, the fluoridated beverage of the present invention can be a milk substitute containing no lactose, yet providing all of the benefits for caries inhibition. Such a composition would comprise the fluoride compound and hydrogen fluoride, the polyprotic acid, as well as the carbohydrate, protein, vitamin A, vitamin C, calcium and vitamin D generally found in a milk/dairy product. The beverage, however, would not contain lactose, and therefore avoid the problems of milk for lactose intolerant individuals.

Yet in another embodiment of the present invention, there is provided oral healthcare products. Such products include toothpaste, mouthwash, dental floss, whitening strips and toothbrush, as well as fluoride drops, capsules, topical compositions or lollipops/suckers. All of the foregoing products are made using the fluoridated solution of the present invention.

Among other things, the present invention is based upon the discovery that one maximizes caries inhibition when one combines a fluoride compound with a polyprotic acid, and a second fluoride source which is hydrogen fluoride. The addition of lysine is also preferred for its benefits of improved absorption of fluoride and calcium. The pH of the fluoridated solution is slightly acidic, which increases fluoride activity, and the presence of the hydrogen fluoride as a second fluoride ion source has been found to maintain or maximize that activity. The fluoridated beverage/solution will maintain idealized buffering capacity without the use of or need for carbonation, and can also provide additional minerals, vitamins and amino acids which contribute to the overall health of an individual, while also contributing to the effectiveness of i.e., the fluoridated beverage in inhibiting caries. In essence, when all of the components, the fluoride ions, vitamin C, lysine, calcium, vitamin D, are combined within the same delivery system, the overall delivery system becomes a complete and profound comprehensive nutritional source toward improved dental strength and greater bone differentiation for enhanced bone health. Concurrently, the addition of such minerals, vitamins and amino acids to the fluoridated beverage of the present invention also minimizes the associated diseases associated with the lack thereof in the human body. Therefore, such a water nutritional delivery system is unique and unparalleled when compared to other water systems presently available to the general public.

### DETAILED DESCRIPTION OF THE PREFERRED EMBODIMENTS

The present invention provides one with a fluoridated beverage, preferably water based, which comprises a fluoride compound, a polyprotic acid, and hydrogen and fluoride ions from hydrogen fluoride. The pH of the beverage is slightly acidic, and is preferably in the range of from about 2.0 to about 7.0, and more preferably in the range of from about 3.0 to 6.5, and most preferably from 3.5 to 6.0. The beverage can be marketed in any suitable container, and commercialized as bottled, fluoridated drinking water.

The fluoridated beverage contains a dual fluoride source which comprises a fluoride compound and hydrogen fluoride. When the fluoride compound is dissolved in water, fluoride ions are yielded. The dissolution of the hydrogen fluoride in water yields hydrogen ions and fluoride ions. It has been found that the combination of the two fluoride sources maximizes fluoride availability in the fluoridated beverage.

The fluoride compound of the fluoridated beverage can be any suitable fluoride compound which yields fluoride ions once dissolved in water. The fluoride compound is preferably selected from the group consisting of sodium fluoride, potassium fluoride, magnesium fluoride, sodium monofluorophosphate, potassium monofluorophosphate, magnesium monofluorophosphate, stannous fluoride, calcium fluoride, sodium silica fluoride, various acidulated fluorophosphates, and mixtures thereof. The use of sodium fluoride or stannous fluoride is most preferred.

The fluoridated beverage is prepared by adding hydrogen fluoride to the water, preferably before the fluoride compound discussed above is added to the water. The hydrogen fluoride dissolves and provides both hydrogen ions and fluoride ions. It has been found that when hydrogen fluoride is dissolved in the water, equilibrium is established when only a fraction of the hydrogen fluoride molecules have transferred protons to water molecules, thereby preserving the fluoride ions yielded by the fluoride compound for activity with regard to greater penetration into the tooth structure of one partaking of the beverage. Thus, it is the combination of the hydrogen fluoride and the fluoride compound which allows one to maximize fluoride availability in the fluoridated beverage, with this maximized fluoride activity/availability being maintained and stable for long periods of time. The hydrogen fluoride also has a stabilizing effect on the pH of the beverage solution as well.

The fluoridated beverage of the present invention contains a fluoride ion level ranging from about 0.15 to about 5.0 ppm by weight, more preferably from about 0.3 to 2.0 ppm fluoride ions, and most preferably in the range of from about 0.7 to about 1.2 ppm fluoride ions by weight of the entire fluoridated beverage. It is preferred that not more than 50% of the fluoride ions in the fluoridated beverage are derived from hydrogen fluoride. This fluoride ion level allows one to safely consume the fluoridated beverage while realizing its health benefits.

The fluoridated beverage also contains a polyprotic acid. A polyprotic acid in accordance with the present invention is an acid that contains more than one acidic hydrogen atom. Examples of such acids include sulfuric acid, oxalic acid, sulfurous acid, phosphoric or orthophosphoric acid, carbonic acid or phthalic acid, as well as carboxylic acids such as citric acid, maleic acid, galactouronic acid, tartaric acid, oxalic acid, asparagousic acid, propionic acid, benzoic acid, and other carboxylic acids found in fruit and vegetable products. The most preferred acids for use in the present invention are the weak acids, with citric acid being the most preferred due to its mild acidity and ready availability. Citric fruit juices are readily available and a convenient source of citric acid.

The presence of the acid, in combination with the hydrogen fluoride, lowers the pH of the fluoridated beverage into the acidic range. Preferably, the pH is in the range of from about 2.0 to 7.0, and more preferably in the range of from about 3.0 to 6.5, and most preferably in the range of from about 3.5 to 6.0. The acidity of the fluoridated beverage increases the porosity of the tooth enamel, thereby allowing greater penetration and absorption of fluoride ions into the teeth. This allows for increased fluoride activity, which increased activity will minimize the possibility of fluorosis, which is a potential toxicsity of fluoride, since one will achieve the effects of increased fluoride without having to have an increased amount of fluoride in the fluoridated beverage.

When a stronger acid is chosen for use, such as phosphoric acid or orthophosphoric acid, it is preferred that the acid disassociate in water into a weak acid and a weak base such as phosphoric acid. In the case of phosphoric acid, a phosphate buffer is created by the presence of its weak acid and conjugate base.

One of the advantages of the present invention is that idealized buffering capacity can be maintained, as well as pH stability, without the use of or need for carbonation. As is well known, carbonation can cause the decay and breakdown of tooth enamel, whereas the fluoridated beverage of the present invention minimizes such a problem.

It is also preferred that the fluoridated beverage of the present invention contain vitamins, minerals and essential amino acids, which can improve the overall health of the individual. The presence of such additional ingredients allows for a comprehensive nutritional delivery system which also improves dental strength and greater bone differentiation for enhanced bone health. In a preferred embodiment, the fluoridated beverage would contain one or more of vitamin C, lysine, calcium, vitamin D and a phosphate compound. The presence of vitamin C, lysine and calcium are most preferred as they not only provide added health benefits themselves, as discussed below, but also advantageously impact the fluoride activity of the fluoridated beverage of the present invention.

Vitamin C, or ascorbic acid, is water soluble and an important component for general health. It is essential in wound healing and in the formation of collagen, a protein important in the formation of healthy skin, tendons, bones and supportive tissues. Vitamin C is also readily available from citrus fruits, and their juices. Thus, the inclusion of natural citrus fruits and fruit juices in the fluoridated beverage is a most preferred embodiment. The juice of the citrus fruit would provide vitamin C, as well as citric acid to lower the pH of the beverage solution within the ideal pH range, which as discussed above facilitates the opening of the enamel surface porosity for greater fluoride adsorption into the enamel tooth structure. The use of citrus fruit juices thus can provide an important element of the fluoridated beverage, the advantages of vitamin C, and also add favorable taste and flavor to the beverage.

It is also preferred to include calcium in the fluoridated beverage. Calcium solubility will increase in a lowered pH environment, thereby increasing the amount of calcium that can be present. The presence of the calcium will also increase a calcium-fluoride compound at the dental enamel surface for improved caries prevention. Calcium itself is also a beneficial mineral which strengthens bones and improves the skeletal metabolism. It has also been observed that the presence of calcium tends to counteract possible deleterious dental erosive affects, and thus further minimizes any risks of dental erosion due to the acidity of the fluoridated beverage. The calcium can be added to the fluoridated beverage solution via any well known compound, such as calcium carbonate.

Vitamin D is also a preferred ingredient of the fluoridated beverage as vitamin D is necessary maintenance of bone architecture and normal calcium homeostasis. Vitamin D plays an important role in the gastrointestinal absorption of calcium and phosphate and in bone dissolution and mineralization.

The addition of a phosphate yielding compound to the fluoridated beverage is also preferred. The phosphate can and will act as a buffer, essentially the same affect as a carbonate buffering system. The phosphate buffering system works better under lower pH than that of a carbonate buffering system achieved through carbonation. Another advantage of phosphate buffering as compared to carbonation is that loss of carbon dioxide and carbonation buffering systems will diminish the buffering capacity. Furthermore, the presence of phosphate and calcium together in solution results in crystallization of hydroxyapetite and tooth crystals, thereby increasing calcium bridging at the tooth surface for greater dental protection. The phosphate can be added to the fluoridated system by means of an acid, such as phosphoric acid, or any suitable phosphate compound. Other minerals such as magnesium can also be included in the fluoridated beverage.

Another preferred component for inclusion in the fluoridated beverage is that of an essential amino acid. It has been found that the presence of the essential amino acid tends to increase fluoride activity. The most preferred essential amino acid is that of lysine, as it is non-toxic, very water soluble, and adds no disagreeable taste to the beverage. Due to its beneficial effect on the fluoride activity, the inclusion of lysine in the fluoridated beverage is preferred. It has also been found that lysine improves calcium activity and can therefore provide a double advantage with its presence.

A sweetener can also be added to the composition in an amount to lend a desirable sweet taste to the beverage and will vary depending on the types and amounts of fluorides and other formulating agents added to the subject drinking water compositions. In most cases the sweetener may normally be present in an operative range of about 0.005 percent to about 20.5 percent by weight with respect to the final total weight of the water composition and preferably in an operative range in an amount between 0.5 percent to about 1.5 percent with respect to the total weight of the drinking water composition. Examples of the sweetening agents that can be used in accordance with the present invention are selected from the group consisting of saccharine, sodium saccharine, potassium saccharine, aspartame, xylitol, sorbitol, sodium cyclamate, glycerol and sugar and mixtures thereof.

The fluoridated beverage of the present invention can also contain flavoring agents that are pharmacologically and physiologically nontoxic when used in the suggested amounts. Examples of suitable flavoring agents comprise a member of the group consisting of substances which are liquids, powders, gels, crystals or other substances made by a process of synthesis or similar artifice, extracted, isolated, or otherwise derived, with or without intermediate or final change of identity, from a vegetable, plant, animal, mineral or other source that, when added or applied to the drinking water compositions, is capable of imparting a flavor thereto. Examples of flavoring agents which can be used in the fluoridated drinking water compositions of the present invention are essential oils such as anise oil, cinnamon il, clove oil, menthol, natural and synthetic substances such as grape, lime, mint, chocolate, lemon, strawberry, cherry, etc., and mixtures thereof. In most cases the flavoring agent or mixtures will normally be present in an operative range of about 0.05 percent to about 25 percent by weight with respect to the total weight of the beverage.

Examples of preservatives which can be used in the fluoridated beverage of the present invention are methylparaben, propylparaben, phenyl mercuric nitrate, sodium bisulfite, sodium nitrite, sodium benzoate, benzoic acid, chlorobutanol, ethylenediaminatetractic acid (EDTA), thimerosal, phynylmercuric acetate, disodium calcium EDTA, etc, and mixtures thereof. In most cases the preservative will normally be present in an operative range of 0.05 percent to about 5 percent by weight with respect to the total weight of the composition.

Examples of thickening and emulsifying agents are preferred substances that meet the requirements of the present fluoridated beverage, and in particular spring water compositions, are mucilaginous substances selected from the group consisting of acacia, bentonite, carrageenan moss extractive of sodium, potassium and calcium salt, methyl cellulose, sodium carboxymethyl cellulose, hydroxyethylcellulose, hydroxypropylcellulose and the salts of sodium, calcium and potassium of the cellulose compounds, magnesium aluminum silicate, silica acrogel, gelatin and gelatin derivatives of which gelatin is the main radical, hydrolyzed polyvinyl acetate, hydrolyzed cellulose esters such as cellulose acetate hydrolyzed to an acetyl content of 19 to 26 percent, polyvinyl alcohol, a vinyl alcohol polymer containing urethane carboxylic acid groups, polyethylene oxide, colloidal albumen, naturally occurring or synthetic alginates such as salts of water soluble metals of sodium, potassium and magnesium, polyethylene glycols, propylene glycol alginates, etc. Various combinations of gelling or nongelling agents and activators such as sterculia gum, tragacanth, starch, guar gum and xanthin gum, and mixtures thereof may be used. The concentration of the thickening and emulsifying agent which may be used in the compositions of the present invention is present in the amount from about 0.00 percent to about 10 percent by weight with respect to the weight of the total weight of the composition.

The most advantageous coloring agents for use in the fluoridated drinking water of the present invention are food, drug and cosmetic color additives that are presently certified under the Food, Drug & Cosmetic Act including dyes and colors such as FD & C and D & C red, yellow, green, blue, orange and aluminum and calcium lakes thereof may be used. It must be understood that multiple combinations of colors may be employed to obtain the desired hue of the final composition, or the fluoridated beverage compositions may be desired to be allowed to remain its natural colorless state without artificial coloration.

The fluoridated beverages of the present invention are generally made by dispersing a selected amount of a fluoride compound in water. Generally, the water has not been exposed to a fluoridation process, but is generally obtained directly from a natural spring, or has been microfiltered or distilled. The fluoride compounds can be added via solution or in a powdered mixture, or in any manner suitable for dispersing the desired amount of fluoride ions into the water. Another method would involve using a removable and replaceable cartridge made of micro porous resonance material and containing therein a fluoride compound, either in a dry or liquid state, which for a compound may be dependent upon its solubility potential to release fluoride in the desired amount into the beverage solution. The cartridge may be such that it can be attached to an existing water treatment device or it may be built into the water treatment device as one integral unit.

In a preferred embodiment, the hydrogen fluoride is first added to the water, since it will create a slightly acidic solution, followed by the addition of the fluoride compound and the remaining components. At least the hydrogen fluoride, the fluoride compound and a polyprotic acid is added to create the fluoridated beverage. Additional components, as discussed above, can be preferably added, such as lysine, vitamin C, calcium, and vitamin D and/or a phosphate compound. Other additional components which can be added to the water, either after the fluoride compounds have been added, or before, include sweeteners, flavoring agents, thickening and emulsifying agents, preservatives, alcohols, coloring agents, carbonating agents, syrups and juices etc., and other formulating and manufacturing aids. The addition of the various components must be controlled, however, to maintain the total fluoride content in the beverage to the range of from 0.15 to 5 ppm fluoride ions, and most preferably in the range from 0.7 to 1.2 ppm fluoride ions in the beverage. The amount of hydrogen fluoride and polyprotic acid is also controlled so that the pH is in the appropriate range from about 2.0 to 7.0, and most preferably in the range from about 3.5 to 6.0. Once the components have been added, the beverage composition may simply be mixed to create the final beverage. The beverage can then be dispensed into various containers for commercial sale.

In a preferred embodiment, a beverage powdered composition is used for mixing into the water, either before or preferably after hydrogen fluoride has been added. The novel beverage powdered composition comprises a fluoride compound, an essential amino acid, and a polyprotic acid. The powdered composition may further comprise, and preferably does, vitamin C. The fluoride compound is preferably sodium fluoride or stannous fluoride. The polyprotic acid is preferably a weak acid, such as citric acid, which can be obtained from a citrus fruit. It is also preferred that the essential amino acid is lysine, for which the best results have been observed with regard to enhancing fluoride activity in penetration of tooth enamel. Lysine is also non-toxic and adds no color or taste to the beverage. The powdered composition can also comprise, and preferably does, calcium, vitamin D and/or a phosphate compound.

The amount of powdered composition used for mixing into water is such as to achieve the desired concentration of fluoride ions in the beverage, as well as the desired pH of the beverage solution.

In another preferred embodiment, the fluoridated beverage comprises the fluoride compound, hydrogen fluoride and polyprotic acid so as to establish the amount of fluoride ions in the range of from 0.15 to 5 ppm, and most preferably from 0.7 to 1.2 ppm fluoride ions, and a pH in the range of from about 3 to 6.5, and most preferably in the range of from about 3.5 to 6.0, while also comprising carbohydrate, protein, vitamin A, vitamin C, calcium and vitamin D as generally found in a milk/dairy product. An example of such a fluoridated beverage would include the fluoride compound, hydrogen fluoride and polyprotic acid in addition to 5 wt % sodium, 48 wt % of the carbohydrate, 17 to 20 wt % of a protein, 10 to 12 wt % of vitamin A, 4 to 5 wt % of vitamin C, 30% calcium and 25% vitamin D, based upon the total weight of these additional components. Such a beverage can be a milk substitute as it contains no lactose, thereby avoiding the problems of dairy products for lactose intolerant individuals.

In another embodiment of the present invention, a solution prepared by adding a fluoride compound and polyprotic acid to water, e.g., adding the powdered composition of the present invention to water, and then adding hydrogen fluoride, can be used in the preparation of oral healthcare products. In such an instance, the necessary flavoring agents to create a beverage are not necessarily added. Included among the various oral health care products that can be prepared in accordance with the present invention are toothpaste, mouthwash, dental floss, whitening strips, toothbrush, as well as fluoride drops, fluoride capsules, topical fluoride compositions, or lollipops/suckers. All of the foregoing oral healthcare products can be used successfully to help promote good oral care of the teeth.

The toothpaste can be prepared by utilizing a paste that is typically employed in making toothpaste, but in preparing the paste the solution of the present invention comprising water, a fluoride compound, hydrogen fluoride and a polyprotic acid is acid. The presence of lysine, and even more preferably the presence of calcium are also preferred. This solution is used in mixing the paste, with the final toothpaste product then being prepared using conventional commercial procedures.

The solution of the present invention can also be a mouthwash or dental rinse. The solution can be bottled as a mouthwash or dental rinse and sold as such, or coloring or flavoring agents can be added.

The solution of the present invention comprising water, a fluoride compound, hydrogen fluoride and a polyprotic acid, as well is preferably lysine and calcium, can also be used in treating dental floss or whitening strips, as well as the bristles of a toothbrush. Such treated healthcare products can then be sold commercially, as is conventional.

Treatment of teeth can also be achieved by using the solution of the present invention and adding thereto a hardening agent to prepare a lollipop or sucker. One sucking on the lollipop would thereby gain the benefits of fluoride treatment of the teeth, and in particular the benefits of the present invention which preferably also contains lysine and calcium to improve calcium activity. The solution of the present invention can also be used as fluoride drops, fluoride capsules where the solution is placed in a gel capsule or other polymeric capsule, or as a topical treatment for teeth, whether in gel or solution form.

The powdered composition of the present invention, therefore, can be added to water, to which hydrogen fluoride is added, to prepare a solution which can be used to make many oral healthcare products and allow one to enjoy the advantages of improved fluoride treatment. The solution can also be used as a beverage or to make a flavored beverage, which need not be carbonated in order to enjoy stability of the fluoride content. Drinking the beverage, whether a water or flavored beverage, will provide optimal caries inhibition.

The present invention will be further illustrated by the following Examples. The following Examples are meant to be merely illustrative, and are in no manner meant to be limiting.

### EXAMPLE 1

A fluoridated beverage in accordance with the present invention is prepared as follows: To 1,000,000 ml. of water 2.2 mg. sodium fluoride*, 0.6 mg hydrogen fluoride and 9,000 mg. orthophosphoric acid were added and completely dissolved before packaged into plastic containers. The composition contains the following:

| | Weight/Volume | |
|---|---|---|
| Water | 1,000,000 | ml. |
| Sodium Fluoride | 2.2 | mg. |
| Hydrogen Fluoride | 0.6 | mg. |
| Orthophosphoric Acid | 9,000.0 | mg. |
| (Phosphoric Acid) | | |

*2.2 mg. sodium fluoride contains 1 mg. fluoride ion. The pH of this composition is between 3.0 and 3.5.

### EXAMPLE 2

A flavored drinking water was prepared containing the following by mixing the ingredients into the water:

| | Weight % |
|---|---|
| Sodium fluoride | 0.00022 |
| Water, spring pure | 97.19168 |
| Hydrogen Fluoride | 0.00010 |
| Citric Acid | 1.31 |
| Strawberry Flavor | 1.25 |
| Sodium Saccharine | 0.20 |
| Sodium Benzoate | 0.04 |
| FD & C RED #40 | 0.008 |

### EXAMPLE 3

A carbonated beverage composition was prepared containing the following by mixing the ingredients into the water:

| | Weight % |
|---|---|
| Hydrogen Fluoride | 0.00010 |
| Sodium fluoride | 0.00022 |
| Water, purified | 59.78 |
| Sodium Carbonate | 12.45 |
| Orthophosphoric Acid | 1.00 |
| Citric acid | 0.55 |
| Blueberry syrup | 25.55 |
| Sodium saccharine | 0.23 |
| Preservative | 0.43 |
| FD & C Red #40 | 0.006 |
| FD & C Blue #2 | 0.002 |

This composition has a pH of about 5.2
*Blueberry syrup was prepared by adding 5.55 ml. of blueberry flavor into 12.00 ml pure spring water and 8.00 ml. of glycerol. This composition has the ph 5.6.

### EXAMPLE 4

A carbonated beverage composition was prepared containing the following by mixing the ingredients into the water:

| | Weight % |
|---|---|
| Sodium fluoride | 0.00028 |
| Hydrogen Fluoride | 0.00009 |
| Orthophosphoric Acid | 0.98 |
| Water, carbonated, spring pure | 71.77 |
| High fructose corn syrup | 18.96 |
| Caffeine | 0.94 |
| Sucrose caramel color | 6.35 |
| Kola flavor | 0.65 |
| Preservative | 0.35 |

### EXAMPLE 5

A flavored beverage was prepared containing the following by mixing the ingredients into the water:

| | Weight % |
|---|---|
| Stannous fluoride | 0.0004* |
| Hydrogen Fluoride | 0.0002 |
| Water, carbonated, spring pure | 79.18 |
| Citric acid | 3.85 |
| Lime flavor | 0.86 |
| Orange flavor | 0.82 |
| Sodium citrate | 2.25 |
| Vitamins | 12.45 |
| Preservative | 0.36 |
| Sweetener | 0.22 |
| FD & C Yellow #5 | 0.008 |
| FD & C Blue #1 | 0.0015 |

*4.0 mg. stannous fluoride yields 1.0 mg. fluoride ions.

### EXAMPLE 6

A flavored spring pure drinking water was prepared containing the following ingredients by mixing the ingredients into the water:

| | Weight % |
|---|---|
| Sodium monofluorophosphate | 0.00076* |
| Hydrogen Fluoride | 0.00036 |
| Phosphoric Acid | 0.99 |
| Citric Acid | 0.50 |
| Xylitol, sweetener | 0.52 |
| Propylene Glycol Alginate | 0.41 |
| Sodium Benzoate | 0.38 |
| FD & C Red #40 | 0.008 |
| FD & C Blue #1 | 0.0003 |
| Water, purified | 97.03 |

*7.6 mg. sodium monofluorophosphate yield 1.0 mg. fluoride ions.

### EXAMPLE 7

A drinking water composition was prepared containing the following by mixing the ingredients into the water:

| | Weight % |
|---|---|
| Stannous fluoride | 0.0804 |
| Hydrogen Fluoride | 0.0350 |
| Phosphoric Acid | 1.00 |
| Water, spring pure | 98.88 |

### COMPARATIVE EXAMPLE 1

A flavored beverage was prepared containing the following:

| | Weight % |
|---|---|
| Sodium fluoride | 0.000154 |
| Citric Acid | 4.15 |
| Flavor | 1.05 |
| Preservative | 0.45 |
| Sweetener | 0.18 |
| Thickeners | 0.005 |
| Colors | 0.003 |
| Water, carbonated, spring pure | 94.16 |

This composition was prepared by dissolving citric aid, flavor, preservative sweetener, thickener and color into the carbonated water and stirred until completely in solution. Sodium fluoride was then added in the composition with continuous stirring for about 10 minutes. The final pH of the composition was found to be 2.0 with quinhydrone assay and the concentration of fluoride ion 0.7 ppm. with colorimetric titration method. The composition was allowed to remain closed in a plastic bottle for one week and then again the pH and fluoride ion concentration measured with the above methods. It was found that the pH rose slightly to 2.1 and the fluoride ion dropped to 0.5 ppm. Four weeks from the base line, the determinations were again taken and it was discovered that the pH rose to 2.2 and the fluoride ion concentration dropped to 0.3 ppm. and after eight weeks the results were 2.2 pH and 0.15 ppm. concentration.

### EXAMPLE 8

A flavored beverage was prepared containing the following:

| | Weight % |
|---|---|
| Sodium fluoride | 0.000154 |
| Hydrogen Fluoride | 0.00007 |
| Citric Acid | 4.15 |
| Flavor | 1.05 |
| Preservative | 0.45 |
| Sweetener | 0.18 |
| Thickeners | 0.005 |
| Colors | 0.003 |
| Water, carbonated, spring pure | 94.16 |

This composition was prepared similarly to Comparative Example 1 with the addition of hydrogen fluoride. The final pH of the composition was found to be 2.0 with quinhydrone assay and the concentration of fluoride ion 0.7 ppm. with colorimetric titration method. The composition was allowed to remain closed in a plastic bottle for one week and then again the pH and fluoride ion concentration measured with the above methods. It was found that the pH and the fluoride ion remained the same. After four weeks and eight weeks from the base line, the pH and the fluoride ion remained the same. This demonstrates the beneficial effects of using the dual fluoride source where one source is hydrogen fluoride. The stability of the beverage and its activity and fluoride availability are maximized.

As illustrated, the present invention, therefore, provides a fluoridated water beverage and method for making bottled drinking water compositions which provide fluoride for the control and prevention of dental caries development comprising preferably pure spring water or filter-purified water and (a) water soluble or water miscible fluoride yielding compound, (b) hydrogen fluoride in concentration of not more than 50% of the concentration of the fluoride yielding compound and (c) a polyprotic acid, in such concentrations that they will provide optimal protection against dental caries.

The present invention also provides a method for making bottled fluoridated, lysine, vitamin C drinking compositions which when taken internally provide optimal protection against tooth decay and improved skeletal metabolism, comprising water and pharmaceutically and chemically compatible harmless formulating aids selected from a group consisting of natural citrus fruits, preservatives, vitamins, and other suitable additives and mixtures thereof wherein water, fluoride, lysine, vitamin C and/or calcium is preferably part of the composition.

The present invention obviates the problems of the prior art by providing fluoridated beverages suitable for consumption containing fluoride ions in such low concentrations that deleterious effects are avoided, yet which permit enhanced fluoride activity, particularly with regard to tooth decay prevention.

While the present invention has been described in relation to its preferred embodiments, it is to be understood that various modifications thereof will become apparent to those skilled in the art upon reading the specification. Therefore, it is to be understood that the invention disclosed herein is intended to cover such modifications as fall within the scope of the claims appended hereto.

What is claimed is:

1. A composition in powder form comprising a fluoride compound, an essential amino acid and a polyprotic acid.

2. The composition of claim 1, further comprising vitamin C.

3. The composition of claim 2, wherein the fluoride compound is selected from the group consisting of sodium fluoride, potassium fluoride, magnesium fluoride, sodium monofluorophosphate, potassium monofluorophosphate, magnesium monofluorophosphate, stannous fluoride, calcium fluoride, sodium silicofluoride, acidulated fluorophosphates, and mixtures thereof.

4. The composition of claim 2, wherein the essential amino acid comprises lysine.

5. The composition of claim 4, further comprising calcium.

6. The composition of claim 1, wherein the polyprotic acid is a carboxylic acid.

7. The composition of claim 1, wherein the acid is citric acid.

8. The composition of claim 2, wherein the composition comprises citric fruit.

9. The composition of claim 2, further comprising vitamin D.

10. The composition of claim 2, further comprising calcium.

11. The composition of claim 2, further comprising a phosphate compound.

12. The composition of claim 2, wherein the fluoride compound is selected from the group consisting of sodium fluoride, and stannous fluoride, the essential amino acid is comprised of lysine, and the acid is citric acid.

13. A fluoridated solution comprising water, a fluoride compound, hydrogen fluoride, and a polyprotic acid.

14. The fluoridated solution of claim 13, further comprising lysine.

15. The fluoridated solution of claim 14, further comprising calcium.

16. The fluoridated solution of claim 13, wherein the solution is not carbonated.

17. The fluoridated solution of claim 13, wherein the solution is a fluoridated beverage for human consumption.

18. The fluoridated beverage of claim 17, wherein the fluoride compound is selected from the group consisting of sodium fluoride, potassium fluoride, magnesium fluoride, sodium monofluorophosphate, potassium monofluorophosphate, magnesium monofluorophosphate, stannous fluoride, calcium fluoride, sodium silicofluoride, acidulated fluorophosphates, and mixtures thereof.

19. The fluoridated beverage of claim 17, wherein the acid is citric acid.

20. The fluoridated beverage of claim 17, further comprising an essential amino acid.

21. The fluoridated beverage of claim 17, wherein the beverage is not carbonated.

22. The fluoridated beverage of claim 20, wherein the essential amino acid comprises lysine.

23. The fluoridated beverage of claim 17, further comprising vitamin C.

24. The fluoridated beverage of claim 23, further comprising vitamin D, calcium and/or a phosphate compound.

25. The fluoridated beverage of claim 17, wherein the fluoride compound is selected from the group consisting of sodium fluoride and stannous fluoride, the acid is citric acid, the composition further comprises lysine and vitamin C.

26. The fluoridated beverage of claim 17, wherein the composition further comprises sodium, a carbohydrate, protein, vitamin A, vitamin C, calcium and vitamin D.

27. The fluoridated beverage of claim 26, wherein the amounts in percent by weight, excluding water, of the components include 5 wt % sodium, 4 to 8 wt % carbohydrate, 17 to 20 wt % protein, 10 to 12 wt % vitamin A, 4 to 5% vitamin C, 30% calcium and 25% vitamin D.

28. The composition of claim 27, further comprising lysine.

29. A process for preparing the fluoridated beverage of claim 17, which comprises
(a) providing water, and
(b) adding to the water hydrogen fluoride, at least one additional fluoride compound, and a polyprotic acid.

30. The process of claim 29, wherein the process involves adding citric juice to the water.

31. The process of claim 29, wherein an essential amino acid is added to the water.

32. The process of claim 29, wherein vitamin C is added to the water.

33. The process of claim 29, wherein vitamin D, calcium and/or phosphate is added to the water.

34. The process of claim 29, wherein lysine is also added to the water.

35. A toothpaste composition comprised of paste suitable for cleaning teeth prepared by utilizing the solution of claim 13 to make the paste.

36. A toothpaste composition comprised of paste suitable for cleaning teeth prepared by utilizing the solution of claim 14 to prepare the paste.

37. The toothpaste composition of claim 36, wherein the solution used to prepare the paste further comprises calcium.

38. A mouthwash comprised of the solution of claim 13.

39. A mouthwash comprised of the solution of claim 14.

40. The mouthwash of claim 39, wherein the solution further comprises calcium.

41. A dental floss which has been treated with the solution of claim 13.

42. A dental floss which has been treated with the solution of claim 14.

43. The dental floss of claim 42, wherein the solution used to treat the dental floss comprises calcium.

44. A whitening strip which has been treated with the solution of claim 13.

45. A whitening strip which has been treated with the solution of claim 14.

46. The whitening strip of claim 45, wherein the solution used in the treatment further comprises calcium.

47. A toothbrush comprised of bristles treated with the solution of claim 13.

48. A toothbrush comprised of bristles treated with the solution of claim 14.

49. A capsule containing the fluoridated solution of claim 13.

50. A topical composition for application to the skin or teeth, comprising the fluoridated solution of claim 13.

51. A dropper bottle containing the solution of claim 13 for application to the teeth.

52. A lollipop comprised of the solution of claim 13 further comprising a gelling or hardening agent.

53. A lollipop comprised of the solution of claim 14 further comprising a gelling or hardening agent.
